(19) United States
(12) Patent Application Publication
(10) Pub. No.: US 2013/0006301 A1
(43) Pub. Date: Jan. 3, 2013

(54) Material For Treating Lumen Defects

(75) Inventors: Timothy Sargeant, Guilford, CT (US); Jonathan Thomas, New Haven, CT (US)

(73) Assignee: Tyco Healthcare Group LP, Mansfield, MA (US)

(21) Appl. No.: 13/481,978

(22) Filed: May 29, 2012

Related U.S. Application Data
(60) Provisional application No. 61/502,062, filed on Jun. 28, 2011.

(51) Int. Cl. A61B 17/03

(52) U.S. Cl. 606/214; 606/213

(57) ABSTRACT

A method of treating tissue defects includes placing at least one polymeric sheet over a tissue defect, in embodiments a lumen defect, to define a defect volume and filling the defect volume with at least one hydrogel precursor including at least one reactive functional group.

### CROSS-REFERENCE TO RELATED APPLICATIONS

This application claims the benefit of and priority to U.S. Provisional Patent Application No. 61/502,062 filed Jun. 28, 2011, the entire disclosure of which is incorporated by reference herein.

### TECHNICAL FIELD

The present disclosure relates to the treatment of lumen defects. More particularly, the present disclosure relates to a wound treatment system including polymer sheets to seal a lumen defect and define a defect volume, and a scaffold to fill the defined defect volume.

### BACKGROUND

A number of diseases result in chronic wounds and tissue death whereby the body cannot heal itself, such as diabetic foot ulcers and holes in body lumens like the esophagus and bowel. These ailments can be critical and cause pain and suffering for the patient. For example, a hole formed in the esophagus (i.e., an esophageal fistula) may cause infection or sepsis, and may not allow the patient to pass food and drink, thereby necessitating a feeding tube.

Current techniques for repairing wounds or lumen defects include, for example, the use of bulking agents such as collagen, dermis, and cadaver allograft material, or the use of autografts. These techniques, however, present healing problems such as limited or delayed incorporation into tissue; mechanical instability or inconsistency as the anatomical site, donor age, and tissue processing conditions may vary; decreased strength in the post-operative period; slow revascularization, recellularization, and/or tissue remodeling; and in the case of allograft material, risk of disease transmission and/or tissue rejection.

Improved materials and methods of treating surface wounds and lumen defects thus remain desirable.

### SUMMARY

Materials suitable for treating defects in tissue, in embodiments lumen defects, are provided, as well as kits including these materials and methods for their use. In embodiments, a method of the present disclosure includes placing a first polymeric sheet over one side of a lumen defect, placing a second polymeric sheet over an opposite side of the lumen defect to define a defect volume, and filling the defect volume with at least one hydrogel precursor including at least one reactive functional group.

The polymeric sheets used to define the defect volume may be non-porous, porous, or combinations thereof, including a composite of non-porous and porous layers. In embodiments, the polymeric sheets may include a tissue facing surface possessing at least one pendant functional group for chemically binding the polymeric sheet to tissue. In other embodiments, the polymeric sheets may include a tissue facing surface including grip members for mechanically binding the polymeric sheet to tissue.

The at least one hydrogel precursor may, in embodiments, include an electrophilic group, a nucleophilic group, or combinations thereof. In embodiments, the at least one hydrogel precursor is placed into a delivery device for introduction into the defect volume. Thus, a method of the present disclosure may further include ejecting the at least one hydrogel precursor from the delivery device through at least one of the first polymeric sheet and the second polymeric sheet.

Bioactive agents may also be included as part of the polymeric sheets used to define the defect volume and/or the at least one hydrogel precursor.

### BRIEF DESCRIPTION OF THE DRAWINGS

FIGS. 1A-1C schematically illustrate a method of using a wound treatment system to treat a surface wound in accordance with an embodiment of the present disclosure;

FIGS. 2A-2C schematically illustrate a method of using a wound treatment system to treat a lumen defect in accordance with an embodiment of the present disclosure; and

FIGS. 3A-3D schematically illustrate a method of using a wound treatment system to treat a lumen defect in accordance with another embodiment of the present disclosure.

### DETAILED DESCRIPTION

In accordance with the present disclosure, a two component wound treatment system is utilized to seal, fill, and treat a tissue defect. The first component creates an artificial surface or interface through which the second component may be placed. The first component is utilized to define the tissue defect volume and seal the surface thereof, and the second component fills the defect volume defined by the first component.

The two component system may be used in a variety of surgical and wound applications involving tissue defects. As used herein, a “tissue defect” may include any breakdown of tissue from a normal, healthy state, including surface wounds and lumen defects. This breakdown may be due to internal factors such as degenerative disease, or external factors such as injury. Any variation from the normal structure of a tissue may be a “tissue defect.” Thus, the two component wound treatment system of the present disclosure may be used to fill voids as a tissue filler, bone filler, or filler for soft/hard tissue interfaces; to promote tissue growth as a tissue scaffold; and/or to deliver bioactive agents and/or cells to a tissue defect or lesion.

The first component of the wound treatment system of the present disclosure is a polymeric sheet that is adapted to adhere to tissue and to seal the tissue defect. The first component is dimensioned to surround the tissue defect such that the polymeric sheet may adhere to the surrounding healthy tissue to create a seal around the tissue defect. The sheet may be a film, foam, mesh, patch, or other substrate adapted to adhere and seal tissue. In embodiments, the first component may be a composite of sheets, including porous and/or non-porous layers of fibers, foams, and/or films.

The first component may be fabricated from any biodegradable and/or non-biodegradable polymer that can be used in surgical procedures. The term “biodegradable” as used herein is defined to include both bioabsorbable and bioresorbable materials. By biodegradable, it is meant that the material decomposes, or loses structural integrity under body conditions (e.g., enzymatic degradation or hydrolysis), or is broken down (physically or chemically) under physiologic conditions in the body, such that the degradation products are excretable or absorbable by the body. Absorbable materials are absorbed by biological tissues and disappear in vivo at the end of a given period, which can vary, for example, from hours to several months, depending on the chemical nature of the material. It should be understood that such materials include natural, synthetic, bioabsorbable, and/or certain non-absorbable materials, as well as combinations thereof.

Representative natural biodegradable polymers which may be used to form the first component include: polysaccharides such as alginate, dextran, chitin, chitosan, hyaluronic acid, cellulose, collagen, gelatin, fucans, glycosaminoglycans, and chemical derivatives thereof (substitutions and/or additions of chemical groups including, for example, alkyl, alkylene, amine, sulfate, hydroxylations, carboxylations, oxidations, and other modifications routinely made by those skilled in the art); catgut; silk; linen; cotton; and proteins such as albumin, casein, zein, silk, soybean protein; and combinations such as copolymers and blends thereof, alone or in combination with synthetic polymers.

Synthetically modified natural polymers which may be used to form the first component include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and combinations thereof.

Representative synthetic biodegradable polymers which may be utilized to form the first component include polyhydroxy acids prepared from lactone monomers (such as glycolide, lactide, caprolactone, ε-caprolactone, valerolactone, and δ-valerolactone), carbonates (e.g., trimethylene carbonate, tetramethylene carbonate, and the like), dioxanones (e.g., 1,4-dioxanone and p-dioxanone), 1,dioxepanones (e.g., 1,4-dioxepan-2-one and 1,5-dioxepan-2-one), and combinations thereof. Polymers formed therefrom include: polylactides; poly(lactic acid); polyglycolides; poly(glycolic acid); poly(trimethylene carbonate); poly(dioxanone); poly(hydroxybutyric acid); poly(hydroxyvaleric acid); poly(lactide-co-(ε-caprolactone-)); poly(glycolide-co-(ε-caprolactone)); polycarbonates; poly(pseudo amino acids); poly(amino acids); poly(hydroxyalkanoate)s such as polyhydroxybutyrate, polyhydroxyvalerate, poly(3-hydroxybutyrate-co-3-hydroxyvalerate), polyhydroxyoctanoate, and polyhydroxyhexanoate; polyalkylene oxalates; polyoxaesters; polyanhydrides; polyester anyhydrides; polyortho esters; and copolymers, block copolymers, homopolymers, blends, and combinations thereof.

Synthetic degradable polymers also include hydrophilic vinyl polymers expanded to include phosphorylcholines such as 2-methacryloyloxyethyl phosphorylcholine, hydroxamates, vinyl furanones and their copolymers, and quaternary ammonia; as well as various alkylene oxide copolymers in combination with other polymers such as lactones, orthoesters, and hydroxybutyrates, for example.

Other biodegradable polymers include polyphosphazenes; polypropylene fumarates; polyimides; polymer drugs such as polyamines; perfluoroalkoxy polymers; fluorinated ethylene/propylene copolymers; PEG-lactone copolymers; PEG-polyorthoester copolymers; blends and combinations thereof.

Some non-limiting examples of suitable nondegradable materials from which the first component may be made include polyolefins such as polyethylene (including ultra high molecular weight polyethylene) and polypropylene including atactic, isotactic, syndiotactic, and blends thereof polyethylene glycols; polyethylene oxides; polyisobutylene and ethylene-alpha olefin copolymers; fluorinated polyolefins such as fluoroethylenes, fluoropropylenes, fluoroPEGSs, and polytetrafluoroethylene; polyamides such as nylon, Nylon 6, Nylon 6,6, Nylon 6,10, Nylon 11, Nylon 12, and polycaprolactam; polyamines; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; vinyl halide polymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as polyvinyl acetate; etheylene-methyl methacrylate copolymers; acrylonitrile-styrene copolymers; ABS resins; ethylene-vinyl acetate copolymers; alkyd resins; polycarbonates; polyoxymethylenes; polyphosphazine; polyimides; epoxy resins; aramids; rayon; rayon-triacetate; spandex; silicones; and copolymers and combinations thereof.

The material forming the first component, in embodiments in the form of a polymeric sheet, may be crosslinked with a crosslinking agent to enhance the mechanical strength of the first component. Crosslinking agents are within the purview of those skilled in the art and include, for example, calcium salts such as hydroxyapatite; aldehyde crosslinking agents such as glutaraldehyde; isocyanate crosslinking agents such as hexamethylene diisocyanate; carbodiimide crosslinking agents such as 1-ethyl-3-(3-dimethylaminopropyl) carbodiimide hydrochloride; polyepoxy crosslinking agents such as ethylene glycol diglycidyl ether; and transglutaminase.

In embodiments, initiators may be utilized to crosslink the first component. Such initiators include, but are not limited to, thermal initiators, photoactivatable initiators, oxidation-reduction (redox) systems, free radical initiators, radiation, thermal initiating systems, combinations thereof, and the like. In embodiments, suitable sources of radiation include heat, visible light, ultraviolet (UV) light, gamma ray, electron beam, combinations thereof, and the like. In embodiments, photoinitiators may also be used. Such photoinitiators include, but are not limited to, free radical initiators, redox initiators such as ferrous-bromate, ammonium persulfate/acetic acid, ammonium persulfate-tetramethyl diamine, potassium persulfate/VA 044 (commercially available from Wako Chemicals Inc., Richmond Va.), and the like. UV light may also be used with dye mediated photooxidation, glutaraldehyde crosslinking, dexamethylene diisocyanate crosslinking, carbodiimide crosslinking, combinations thereof, and the like.

At least a portion of the first component, in embodiments in the form of a polymeric sheet, may include at least one pendant functional group suitable for interacting with the tissue and/or the second component of the wound treatment systems described herein. The at least one functional group may be on any portion of the polymeric sheet, such as a surface thereof. The functional group capable of binding to tissue may bind to amines, carboxyl groups, hydroxyl groups, or any other chemistry present on the tissue surface to which the first component is to be attached. Such groups include compounds possessing chemistries having some affinity for tissue.

For amine binding reactions, for example, isothiocyanates, isocyanates, acyl azides, N-hydroxysuccinimide (NHS) and sulfo-NHS esters, sulfonyl chlorides, aldehydes and glyoxals, epoxides and oxiranes, carbonates, arylating agents, imidoesters, carbodiimides, and anhydrides may be utilized. For carboxyl binding reactions, for example, diazoalkanes and diazoacetyl compounds may be utilized, as well as carbonyldiimidazoles, carbodiimides, and NHS, which convert carboxylic acid into a reactive intermediate which is susceptible to reaction with amines or alcohols. For hydroxyl binding reactions, for example, epoxides and oxiranes, carbonyldiimidazoles, disuccinimidyl carbonate and hydroxysuccinimidyl chloroformate, alkyl halogens, isocyanates, and methacryloyl or acryloyl chloride may be utilized, as well as oxidation with periodate or enzymatic oxidation.

It is contemplated by the present disclosure that the functional groups may be the same or different at each occurrence. Thus, the polymeric sheet may have two or more different functional groups for binding to tissue.

The functional groups may be positioned on or near the surface of the polymeric sheet in any suitable manner. For example, the polymeric sheet may be formed from materials which naturally position functional groups toward the outer surface thereof. In other examples, the polymeric sheet may be surface-modified to covalently attach the functional groups thereto. In still other examples, the polymeric sheet may be coated with an additional layer of material, which includes the pendant functional groups necessary to interact with the tissue and/or the second component of the wound treatment systems described herein.

In some embodiments, a coating process for introducing functional groups into the first component includes surface treatment of the polymeric sheet in order to promote adhesion of the coating to the surface of the polymeric sheet. The surface of the polymeric sheet can be treated using plasma, physical or chemical vapor deposition, pulsed laser ablation deposition, surface modification, or any other means within the purview of those skilled in the art to activate the surface of the polymeric sheet with a functionalized coating. In other embodiments, a suitable treatment may include the use of a primer such as a cross-linkable compound. In yet other embodiments, one or more deposition treatments could be used alone or in conjunction with a primer to achieve the desired association of a functionalized coating with the polymeric sheet.

Additionally, or alternatively, the first component may include mechanical means for binding to tissue. In embodiments, the polymeric sheet may be a mesh including mechanical grips or hooks to achieve, or enhance, adhesivity to tissue. Examples of such meshes include, for example, PARIETEX PROGRIP™ self-fixating mesh, commercially available from Covidien.

The second component of a wound treatment system of the present disclosure is a scaffold that fills the tissue defect volume defined by the first component. The scaffold may be a hydrogel, putty, or other filler material which may serve as a space filler and matrix for tissue formation. The scaffold includes structure upon, or within, which the desired cells may grow in order to regenerate the desired tissue. In embodiments, the second component may be capable of reacting with the functional group(s) of the first component to bond thereto. In embodiments, the second component may be porous.

The second component may include at least one hydrogel precursor suitable for forming a hydrogel material. At least one of the hydrogel precursors of the second component may be capable of reacting with the pendant functional group(s) of the first component and/or surrounding tissue within which the second component is placed, to improve the adhesion of the first component and prevent delamination and/or cyst formation at the treatment site. The reactive chemistry of the second component may be the same, or different, than that of the first component. The hydrogel precursor may be, e.g., a monomer or a macromer. The hydrogel precursor may be a solid or a liquid. One type of precursor may have a reactive functional group that is an electrophile or a nucleophile. Electrophiles react with nucleophiles to form covalent bonds. Covalent crosslinks or bonds refer to chemical groups formed by reaction of functional groups on different materials that serve to covalently bind the different materials to each other. In certain embodiments, a first set of electrophilic functional groups on a first precursor may react with a second set of nucleophilic functional groups on a second precursor. When the precursors are mixed in an environment that permits reaction (e.g., as relating to pH or solvent), the functional groups react with each other to form covalent bonds. The precursors become crosslinked when at least some of the precursors can react with more than one other precursor. For instance, a precursor with two or more functional groups of a first type may be reacted with a crosslinking precursor that has two or more functional groups of a second type capable of reacting with the first type of functional groups.

The hydrogel may be formed from single or multiple precursors. For example, where the hydrogel is formed from multiple precursors, for example two precursors, the precursors may be referred to as a first and a second hydrogel precursor. The terms “first hydrogel precursor” and “second hydrogel precursor” each are meant to include any of a polymer, functional polymer, macromolecule, small molecule, or crosslinker that can take part in a reaction to form a network of crosslinked molecules, e.g., a hydrogel.

The term “reactive functional group” as used herein refers to electrophilic or nucleophilic groups capable of reacting with each other to form a bond. Electrophilic functional groups include, for example, N-hydroxysuccinimides (“NHS”), sulfosuccinimides, carbonyldiimidazole, sulfonyl chloride, aryl halides, sulfosuccinimidyl esters, N-hydroxysuccinimidyl esters, succinimidyl esters such as succinimidyl succinates and/or succinimidyl propionates, isocyanates, thiocyanates, carbodiimides, benzotriazole carbonates, epoxides, aldehydes, maleimides, imidoesters, combinations thereof, and the like. In embodiments, the electrophilic functional group is a succinimidyl ester.

As noted above, the present disclosure provides hydrogels which may include an electrophilic precursor, sometimes referred to herein as an electrophilic crosslinker, and a nucleophilic component. In embodiments, the nucleophilic component is a natural component, which may be cross-linked by the electrophilic crosslinker to form a hydrogel. In embodiments, the hydrogel may be biodegradable.

The hydrogel may be formed prior to implantation or may be formed in situ at the time of implantation. The components for forming hydrogels on, or in, tissues may include, for example, in situ forming materials. The in situ forming material may include a single precursor or multiple precursors that form “in situ”, meaning formation occurs at a tissue in a living animal or human body. In general, this may be accomplished by having a precursor that can be activated at the time of application to create, in embodiments, a hydrogel. Activation can be through a variety of methods including, but not limited to, environmental changes such as pH, ionicity, temperature, etc.

In some embodiments, as discussed further below, the hydrogel itself may include a natural component such as collagen, gelatin, hyaluronic acid, combinations thereof, and the like, and thus the natural component may be released at the site of implantation as the hydrogel degrades. The term “natural component” as used herein includes polymers, compositions of matter, materials, combinations thereof, and the like, which can be found in nature or derived from compositions/organisms found in nature. Natural components also may include compositions which are found in nature but can be synthesized by man, for example, using methods to create natural/synthetic/biologic recombinant materials, as well as methods capable of producing proteins with the same sequences as those found in nature, and/or methods capable of producing materials with the same structure and components as natural materials, such as synthetic hyaluronic acid, which is commercially available, for example, from Sigma Aldrich.

The hydrogel precursors, e.g., the electrophilic hydrogel precursors, may have biologically inert and water soluble cores. When the core is a polymeric region that is water soluble, suitable polymers that may be used include: polyethers, for example, polyalkylene oxides such as polyethylene glycol (“PEG”), polyethylene oxide (“PEO”), polyethylene oxide-co-polypropylene oxide (“PPO”), co-polyethylene oxide block or random copolymers, and polyvinyl alcohol (“PVA”); poly(vinyl pyrrolidinone) (“PVP”); poly(amino acids); poly (saccharides) such as dextran, chitosan, alginates, carboxymethylcellulose, oxidized cellulose, hydroxyethylcellulose, and hydroxymethylcellulose; hyaluronic acid; and proteins such as albumin, collagen, casein, and gelatin. Other suitable hydrogels may include components such as methacrylic acid, acrylamides, methyl methacrylate, hydroxyethyl methacrylate, combinations thereof, and the like. In embodiments, combinations and components of the foregoing polymers may be utilized.

The polyethers, and more particularly poly(oxyalkylenes) or poly(ethylene glycol) or polyethylene glycol, may be utilized in some embodiments. When the core is small in molecular nature, any of a variety of hydrophilic functionalities can be used to make the first and second hydrogel precursors water soluble. For example, functional groups like hydroxyl, amine, sulfonate and carboxylate, which are water soluble, may be used to make the precursor water soluble. For example, the n-hydroxysuccinimide (“NHS”) ester of subaric acid is insoluble in water, but by adding a sulfonate group to the succinimide ring, the NHS ester of subaric acid may be made water soluble, without affecting its reactivity towards amine groups. In embodiments, the precursor having electrophilic functional groups may be a PEG ester.

As noted above, each of the first and second hydrogel precursors may be multifunctional, meaning that it may include two or more electrophilic or nucleophilic functional groups, such that, for example, a nucleophilic functional group on the first hydrogel precursor may react with an electrophilic functional group on the second hydrogel precursor to form a covalent bond. At least one of the first or second hydrogel precursors includes more than two functional groups, so that, as a result of electrophilic-nucleophilic reactions, the precursors combine to form cross-linked polymeric products.

A macromolecule having electrophilic functional groups may be multi-armed. For example, the macromolecule may be a multi-armed PEG having four, six, eight, or more arms extending from a core. The core may be the same or different from the macromolecule forming the arms. For example, the core may be PEG and the multiple arms may also be PEG. In embodiments, the core may be a natural polymer.

The molecular weight (MW) of the electrophilic crosslinker may be from about 2,000 to about 100,000 daltons (Da); in embodiments from about 10,000 to about 40,000 Da. Multi-arm precursors may have a molecular weight that varies depending on the number of arms. For example, an arm having a 1000 Da of PEG has enough CH₂CH₂O groups to total at least 1000 Da. The combined molecular weight of an individual arm may be from about 250 to about 25,000 Da; in embodiments from about 1,000 to about 3,000 Da; in embodiments from about 1,250 to about 2,500 Da. In embodiments, the electrophilic crosslinker may be a multi-arm PEG functionalized with multiple NHS groups having, for example, four, six or eight arms and a molecular weight from about 5,000 to about 25,000 Da. Other examples of suitable precursors are described in U.S. Pat. Nos. 6,152,943; 6,165,201; 6,179,862; 6,514,534; 6,566,406; 6,605,294; 6,673,093; 6,703,047; 6,818,018; 7,009,034; and 7,347,850, the entire disclosures of each of which are incorporated herein by reference.

The electrophilic precursor may be a cross-linker that provides an electrophilic functional group capable of bonding with nucleophiles on another component, in embodiments a natural component. The natural component may be endogenous to the patient to which the electrophilic crosslinker is applied, or may be exogenously applied.

In embodiments, one of the precursors may be a natural component possessing nucleophilic groups. Nucleophilic groups which may be present include, for example, —NH₂, —SH, —OH, —PH₂, and —CO—NH—NH₂. Any monomer, macromer, polymer, or core described above as suitable for use in forming the electrophilic precursor may be functionalized with nucleophilic groups to form a nucleophilic precursor. In other embodiments, a natural component possessing nucleophilic groups may be utilized as the nucleophilic precursor.

The natural component may be, for example, collagen, gelatin, blood (including serum, which may be whole serum or extracts therefrom), hyaluronic acid, proteins, albumin, other serum proteins, serum concentrates, platelet rich plasma (prp), combinations thereof, and the like. Additional suitable natural components which may be utilized or added to another natural component, sometimes referred to herein as a bioactive agent, include, for example, stem cells, DNA, RNA, enzymes, growth factors, peptides, polypeptides, antibodies, other nitrogenous natural molecules, combinations thereof, and the like. Other natural components may include derivatives of the foregoing, for example modified hyaluronic acid, dextran, other polysaccharides, polymers and/or polypeptides, including aminated polysaccharides which may be naturally derived, synthetic, or biologically derived. For example, in embodiments hyaluronic acid may be modified to make it nucleophilic.

In embodiments, any of the above natural components may be synthetically prepared, e.g., synthetic hyaluronic acid, utilizing methods within the purview of those skilled in the art. Similarly, in embodiments the natural component could be a natural or synthetic long chain aminated polymer. The natural component may also be modified, i.e., aminated to create a nucleophilic polymer.

The natural component may provide cellular building blocks or cellular nutrients to the tissue that it contacts in situ. For example, serum contains proteins, glucose, clotting factors, minerals, ions, and hormones which may be useful in the formation or regeneration of tissue.

In embodiments, the natural component includes whole serum. In embodiments, the natural component is autologous, i.e., collagen, serum, blood, and the like, from the body where the hydrogel is (or is to be) formed. In this manner, the person or animal in which the hydrogel is to be used may provide the natural component for use in formation of the hydrogel. In such embodiments, the resulting hydrogel is semi-autologous, including a synthetic electrophilic precursor and an autologous/endogenous nucleophilic precursor.

In embodiments, a multifunctional nucleophilic polymer, such as a natural component having multiple amine groups, may be used as a first hydrogel precursor and a multifunctional electrophilic polymer, such as a multi-arm PEG functionalized with multiple NHS groups, may be used as a second hydrogel precursor. In embodiments, the precursors may be in solution(s), which may be combined to permit formation of the hydrogel. Any solutions utilized as part of the in situ forming material system should not contain harmful or toxic solvents. In embodiments, the precursor(s) may be substantially soluble in a solvent such as water to allow application in a physiologically-compatible solution, such as buffered isotonic saline.

In embodiments, a hydrogel may be formed from collagen, or a combination of collagen and/or gelatin, as the natural component, with a multi-functional PEG utilized as a crosslinker. In embodiments, the collagen and/or gelatin may be placed in solution, utilizing a suitable solvent. To this solution, hyaluronic acid may be added along with a high pH buffer. Such a buffer may have a pH from about 8 to about 12, in embodiments from about 8.2 to about 9. Examples of such buffers include, but are not limited to, borate buffers, and the like.

In a second solution, an electrophilic crosslinker, in embodiments a multi-arm PEG functionalized with electrophilic groups such as n-hydroxysuccinimide, may be prepared in a buffer such as Hanks Balanced Salt Solution, Dulbecco's Modified Eagle's Medium, Phosphate Buffered Saline, water, phosphate buffer, combinations thereof, and the like. The electrophilic crosslinker, in embodiments a multi-arm PEG functionalized with n-hydroxysuccinimide groups, may be present in a solution including the above buffer at a concentration from about 0.02 grams/ml to about 0.5 grams/ml, in embodiments from about 0.05 grams/ml to about 0.3 grams/ml.

The two components may be combined, in some embodiments upon introduction in situ, wherein the electrophilic groups on the multi-arm PEG crosslink the amine nucleophilic components of the collagen and/or gelatin. The ratio of natural component to electrophilic component (i.e., collagen:PEG) may be from about 0.1:1 to about 100:1, in embodiments from about 1:1 to about 10:1.

The nucleophilic components, in embodiments the natural components, e.g., collagen, gelatin, and/or hyaluronic acid, may together be present at a concentration of at least about 1.5 percent by weight of the hydrogel, in embodiments from about 1.5 percent by weight to about 20 percent by weight of the hydrogel, in other embodiments from about 2 percent by weight to about 10 percent by weight of the hydrogel. In certain embodiments, collagen may be present from about 0.5 percent to about 7 percent by weight of the hydrogel, in further embodiments, from about 1 percent to about 4 percent by weight of the hydrogel. In another embodiment, gelatin may be present from about 1 percent to about 20 percent by weight of the hydrogel, in further embodiments, from about 2 percent to about 10 percent by weight of the hydrogel. In yet another embodiment, hyaluronic acid and collagen combined as the natural component(s) may be present from about 0.5 percent to about 8 percent by weight of the hydrogel, in further embodiments, from about 1 percent to about 5 percent by weight of the hydrogel. It is also envisioned that the hyaluronic acid may not be present as a “structural” component, but as more of a bioactive agent. For example, hyaluronic acid may be present in solution/gel in concentrations as low as 0.001 percent by weight of the solution/gel and have biologic activity.

The electrophilic crosslinker may be present in amounts of from about 0.5 percent by weight to about 20 percent by weight of the hydrogel, in embodiments from about 1.5 percent by weight to about 15 percent by weight of the hydrogel.

Hydrogel materials may be formed either through covalent, ionic, or hydrophobic bonds. Physical (non-covalent) crosslinks may result from complexation, hydrogen bonding, desolvation, Van der Waals interactions, ionic bonding, combinations thereof, and the like, and may be initiated by mixing two precursors that are physically separated until combined in situ, or as a consequence of a prevalent condition in the physiological environment, including: temperature, pH, ionic strength, combinations thereof, and the like. Chemical (covalent) crosslinking may be accomplished by any of a number of mechanisms, including: free radical polymerization, condensation polymerization, anionic or cationic polymerization, step growth polymerization, electrophile-nucleophile reactions, combinations thereof, and the like.

In some embodiments, hydrogel systems may include biocompatible multi-precursor systems that spontaneously crosslink when the precursors are mixed, but wherein the two or more precursors are individually stable for the duration of the deposition process. In other embodiments, in situ forming materials may include a single precursor that crosslinks with endogenous materials and/or tissues.

The crosslinking density of the resulting biocompatible crosslinked polymer may be controlled by the overall molecular weight between crosslinks of the crosslinker and natural component and the number of functional groups available per molecule. A lower molecular weight between the crosslinks, such as 600 daltons (Da), will give much higher crosslinking density and tighter polymer network as compared to a higher molecular weight, such as 10,000 Da. Elastic gels may be obtained with higher molecular weight natural components with molecular weights of more than 3,000 Da between the crosslinks.

The crosslinking density may also be controlled by the overall percent solids of the crosslinker and natural component solutions. Increasing the percent solids increases the probability that an electrophilic group will combine with a nucleophilic group prior to inactivation by hydrolysis. Yet another method to control crosslink density is by adjusting the stoichiometry of nucleophilic groups to electrophilic groups. A one to one ratio may lead to the highest crosslink density, however, other ratios of reactive functional groups (e.g., electrophile:nucleophile) are envisioned to suit a desired formulation.

The hydrogel thus produced may be bioabsorbable, so that it does not have to be retrieved from the body. Absorbable materials are absorbed by biological tissues and disappear in vivo at the end of a given period, which can vary, for example, from one day to several months, depending on the chemical nature of the material. Absorbable materials include both natural and synthetic biodegradable polymers, as well as bioerodible polymers.

In embodiments, one or more precursors having biodegradable linkages present in between functional groups may be included to make the hydrogel biodegradable or absorbable. In some embodiments, these linkages may be, for example, esters, which may be hydrolytically degraded in physiological solution. The use of such linkages is in contrast to protein linkages that may be degraded by proteolytic action. A biodegradable linkage optionally also may form part of a water soluble core of one or more of the precursors. Alternatively, or in addition, functional groups of precursors may be chosen such that the product of the reaction between them results in a biodegradable linkage. For each approach, biodegradable linkages may be chosen such that the resulting biodegradable biocompatible crosslinked polymer degrades or is absorbed in a desired period of time. Generally, biodegradable linkages may be selected that degrade the hydrogel under physiological conditions into non-toxic or low toxicity products.

Biodegradable gels utilized in the present disclosure may degrade due to hydrolysis or enzymatic degradation of the biodegradable region, whether part of the natural component or introduced into a synthetic electrophilic crosslinker. The degradation of gels containing synthetic peptide sequences will depend on the specific enzyme and its concentration. In some cases, a specific enzyme may be added during the crosslinking reaction to accelerate the degradation process. In the absence of any degradable enzymes, the crosslinked polymer may degrade solely by hydrolysis of the biodegradable segment. In embodiments in which polyglycolate is used as the biodegradable segment, the crosslinked polymer may degrade in from about 1 day to about 30 days depending on the crosslinking density of the network.

Similarly, in embodiments in which a polycaprolactone based crosslinked network is used, degradation may occur over a period of time from about 1 month to about 8 months. The degradation time generally varies according to the type of degradable segment used, in the following order: polyglycolate<polylactate<polytrimethylene carbonate<polycaprolactone. Thus, it is possible to construct a hydrogel with a desired degradation profile, from a few days to months, using a proper degradable segment.

Where utilized, the hydrophobicity generated by biodegradable blocks such as oligohydroxy acid blocks or the hydrophobicity of PPO blocks in PLURONIC™ or TETRONIC™ polymers utilized to form the electrophilic crosslinker may be helpful in dissolving small organic drug molecules. Other properties which will be affected by incorporation of biodegradable or hydrophobic blocks include: water absorption, mechanical properties, and thermosensitivity.

Certain properties of the hydrogel material can be useful, including adhesion to a variety of tissues, desirable setting times to enable a surgeon to accurately and conveniently place the hydrogel materials, high water content for biocompatibility, mechanical strength for use in implants, and/or toughness to resist destruction after placement. Synthetic materials that are readily sterilized and avoid the dangers of disease transmission involved in the use of natural materials may thus be used. Indeed, certain in situ polymerizable hydrogels made using synthetic precursors are within the purview of those skilled in the art, e.g., as used in commercially available products such as FOCALSEAL® (Genzyme, Inc.), COSEAL® (Angiotech Pharmaceuticals), and DURASEAL® (Confluent Surgical, Inc). Other known hydrogels include, for example, those disclosed in U.S. Pat. Nos. 6,656,200; 5,874,500; 5,543,441; 5,514,379; 5,410,016; 5,162,430; 5,324,775; 5,752,974; and 5,550,187.

As noted above, in embodiments a branched multi-arm PEG, sometimes referred to herein as a PEG star, may be included to form a hydrogel of the present disclosure. A PEG star may be functionalized so that its arms include pendant reactive biofunctional groups for biological signaling and/or molecular binding, such as amino acids, peptides, antibodies, enzymes, drugs, affinity binders, thiols, combinations thereof, or other moieties such as bioactive agents in its cores, its arms, or at the ends of its arms. The biofunctional groups may also be incorporated into the backbone of the PEG, or attached to a reactive group contained within the PEG backbone. The binding can be covalent or non-covalent, including electrostatic, thiol mediated, peptide mediated, or using known reactive chemistries, for example, biotin with avidin.

Amino acids incorporated into a PEG star may be natural or synthetic, and can be used singly or as part of a peptide. Sequences may be utilized for cellular adhesion, cell differentiation, combinations thereof, and the like, and may be useful for binding other biological molecules such as growth factors, drugs, cytokines, DNA, antibodies, enzymes, combinations thereof, and the like. Such amino acids may be released upon enzymatic degradation of the PEG star.

These PEG stars may also include functional groups as described above to permit their incorporation into a hydrogel. The PEG star may be utilized as the electrophilic crosslinker or, in embodiments, be utilized as a separate component in addition to the electrophilic crosslinker described above. In embodiments, the PEG stars may include electrophilic groups that bind to nucleophilic groups. As noted above, the nucleophilic groups may be part of a natural component utilized to form a hydrogel of the present disclosure.

In some embodiments a biofunctional group may be included in a PEG star by way of a degradable linkage, including an ester linkages formed by the reaction of PEG carboxylic acids or activated PEG carboxylic acids with alcohol groups on a biofunctional group. In this case, the ester groups may hydrolyze under physiological conditions to release the biofunctional group.

Bioactive agents may be added to the first and/or second component to provide specific biological or therapeutic properties thereto. Any product which may enhance tissue repair, limit the risk of sepsis, and modulate the mechanical properties of the first and/or second components, or specific portion thereof, may be added during the preparation of a component of the wound treatment system or may be coated on the first component, in embodiments a polymeric sheet. In embodiments, agents which may be added include: fucans for antiseptic properties; chitosan and glutaraldehyde crosslinked collagen for their degradation time; and growth factors, peptides, proteins, drugs, and DNA for their tissue properties.

Moreover, the first and/or second component may also be used for delivery of one or more bioactive agents. The bioactive agents may be incorporated into one or both of the first and/or second component during formation thereof, such as by free suspension, liposomal delivery, microspheres, etc., or by coating a surface of the polymeric sheet, or portion thereof, such as by polymer coating, dry coating, freeze drying, applying to a surface of the polymeric sheet, ionically, covalently, or affinity binding to functionalize the degradable components of the wound treatment system. Thus, in some embodiments, at least one bioactive agent may be combined with the first and/or second component during formation to provide release of the bioactive agent during degradation of the first and/or second component. As the first and/or second component degrades or hydrolyzes in situ, the bioactive agents are released. In other embodiments, bioactive agents may be coated onto a surface or a portion of a surface of the polymeric sheet for quick release of the bioactive agent. In embodiments, the polymeric sheet of the first component may act as a diffusion barrier for bioactive agents delivered with, or contained within, the hydrogel.

A bioactive agent as used herein is used in the broadest sense and includes any substance or mixture of substances that have clinical use. Consequently, bioactive agents may or may not have pharmacological activity per se, e.g., a dye. Alternatively a bioactive agent could be any agent that provides a therapeutic or prophylactic effect; a compound that affects or participates in tissue growth, cell growth, and/or cell differentiation; an anti-adhesive compound; a compound that may be able to invoke a biological action such as an immune response; or could play any other role in one or more biological processes. A variety of bioactive agents may be incorporated into a component of the wound treatment system.

Examples of classes of bioactive agents, which may be utilized in accordance with the present disclosure include, for example, anti-adhesives, antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, platelet activating drugs, clotting factors and enzymes. It is also intended that combinations of bioactive agents may be used.

Other bioactive agents, which may be included are: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents, such as coronary vasodilators and nitroglycerin; alkaloids; analgesics; narcotics such as codeine, dihydrocodeinone, meperidine, morphine and the like; non-narcotics, such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents, such as hormonal agents, hydrocortisone, prednisolone, prednisone, non-hormonal agents, allopurinol, indomethacin, phenylbutazone and the like; prostaglandins and cytotoxic drugs; chemotherapeutics; estrogens; antibacterials; antibiotics; anti-fungals; anti-virals; anticoagulants; anticonvulsants; antidepressants; antihistamines; and immunological agents.

Other examples of suitable bioactive agents, which may be included in the first and/or second component include, for example, viruses and cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, α-IFN and γ-IFN); erythropoietin; nucleases; tumor necrosis factor; colony stimulating factors (e.g., GCSF, GM-CSF, MCSF); insulin; anti-tumor agents and tumor suppressors; blood proteins such as fibrin, thrombin, fibrinogen, synthetic thrombin, synthetic fibrin, synthetic fibrinogen; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); bone morphogenic proteins; TGF-B; protein inhibitors; protein antagonists; protein agonists; nucleic acids, such as antisense molecules, DNA, RNA, RNAi; oligonucleotides; polynucleotides; and ribozymes.

It may be desirable to include bioactive agents which promote wound healing and/or tissue growth, including colony stimulating factors, blood proteins, fibrin, thrombin, fibrinogen, hormones and hormone analogs, blood coagulation factors, growth factors, bone morphogenic proteins, TGF-β, IGF, combinations thereof, and the like. In embodiments, the first and/or second component may deliver and/or release biological factors/molecules and/or cells at the site of implantation. Thus, it may assist in native tissue regrowth by providing the surrounding tissue with needed nutrients and bioactive agents.

As noted above, in embodiments in which the second component includes a multi-arm PEG or PEG star, the bioactive agent may be incorporated into the core of the PEG, the arms of the PEG, or combinations thereof. In embodiments, the bioactive agent may be attached to a reactive group in the PEG chain. The bioactive agent may be bound covalently, non-covalently, i.e., electrostatically, through a thiol-mediated or peptide-mediated bond, or using biotin-avidin chemistries and the like.

In embodiments, the bioactive agent may be encapsulated by the hydrogel of the second component. For example, the hydrogel may form polymer microspheres around the bioactive agent. As the hydrogel hydrolyzes in situ, the bioactive components and any added bioactive agents are released. This may provide nutrients from the natural components, as well as bioactive agents, to the surrounding tissue, thereby promoting growth and/or regeneration of tissue.

Various combinations of first and second components may be used to treat a tissue defect in accordance with the present disclosure. For example, any of the polymeric sheet materials and configurations as described above may be combined with any of the second component hydrogels also described above, dependent upon the type of defect to be treated and the properties desired from the wound treatment system.

In accordance with the present disclosure, the first component of the wound treatment system seals and defines a defect volume in a tissue defect. The first component is configured to allow for the passage of the second component therethrough. The first component may be a porous or non-porous polymeric sheet, or composite of sheets, including at least one functional group on a surface thereof for binding to healthy tissue surrounding the tissue defect. Additionally, or alternatively, the first component may include mechanical means for binding to tissue. In embodiments, the at least one functional group is also reactive with the second component of the wound treatment system.

The second component of the wound treatment system of the present disclosure promotes tissue repair in a tissue defect by filling the void of the defect with a tissue specific scaffold which promotes its respective tissue regeneration. The second component also promotes integration with a tissue void by form fitting the defect. The second component may be a single or multi-component hydrogel containing water soluble biopolymers as at least one component. The precursor(s) of the hydrogel may be dissolved to form a solution prior to use, with the solution being delivered to the tissue defect. As used herein, a solution may be homogeneous, heterogeneous, phase separated, or the like. In other embodiments, the precursor(s) may be in an emulsion. Where two solutions are employed, each solution may contain one precursor of the hydrogel forming material which forms upon contact. The solutions may be separately stored and mixed when delivered to tissue.

In a single component system, the precursor, i.e., the electrophile, reacts with natural components of the tissue environment to produce a crosslinked polymeric network. In a multi-component system, the precursors react with each other to form a hydrogel. In embodiments, the precursors are nucleophilic/electrophilic reactive components, such as succinimide and primary amines. In both the single and multi-component hydrogel systems, the hydrogel may crosslink with the first component of the wound treatment system. In embodiments, a biopolymer component of the hydrogel may promote cell attachment and proliferation.

Formulations may be prepared that are suited to make precursor crosslinking reactions occur in situ. In general, this may be accomplished by having a precursor that can be activated at the time of application to a tissue to form a crosslinked hydrogel. Activation can be made before, during, or after application of the precursor to the tissue, provided that the precursor is allowed to conform to the tissue's shape before crosslinking and associated gelation is otherwise too far advanced. Activation includes, for instance, mixing precursors with functional groups that react with each other. Thus, in situ polymerization includes activation of chemical moieties to form covalent bonds to create an insoluble material, e.g., a hydrogel, at a location where the material is to be placed on, within, or both on and within, a patient. In situ polymerizable polymers may be prepared from precursor(s) that can be reacted such that they form a polymer within the patient. Thus precursor(s) with electrophilic functional groups can be mixed or otherwise activated in the presence of precursors with nucleophilic functional groups.

In other embodiments, where electrophilic precursors are used, such precursors may react with free amines in tissue, thereby serving as a means for attaching the hydrogel to tissue.

The crosslinking reaction leading to gelation can occur, in some embodiments within a time from about 1 second to about 5 minutes, in embodiments from about 3 seconds to about 1 minute; persons of ordinary skill in these arts will immediately appreciate that all ranges and values within these explicitly stated ranges are contemplated. For example, in embodiments, the in situ gelation time of hydrogels according to the present disclosure is less than about 20 seconds, and in some embodiments, less than about 10 seconds, and in yet other embodiments less than about 5 seconds.

Embodiments of the present disclosure will now be described, by way of example only, with reference to the accompanying drawings.

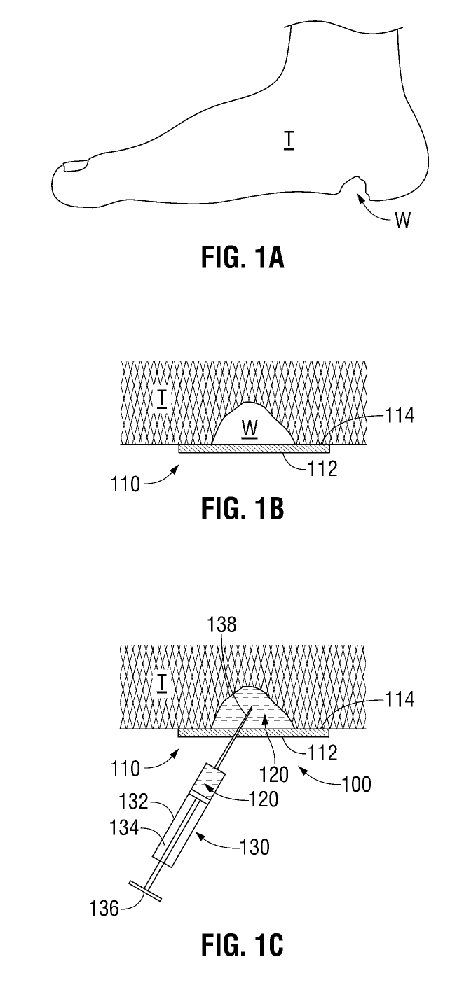

Referring now to FIGS. 1A-1C, a wound treatment system 100 including a first component 110 and a second component 120 is illustrated for repairing a surface wound “W”, i.e., a diabetic foot ulcer, in tissue “T”. After a tissue defect, i.e., a surface wound “W”, has been identified and cleaned, as shown in FIG. 1A, a first component 110 of system 100 may be placed over the wound “W” as illustrated in FIG. 1B. First component 110 is a solid polymeric sheet 112 including at least one functional group 114 on a surface thereof for adhering to tissue “T” surrounding wound “W”. The surface of tissue “T” is sealed with the first component 110 to define a defect volume of wound “W”. A delivery device 130 loaded with second component 120 may be delivered through the first component 100 as illustrated in FIG. 1C. The second component 120 is a hydrogel formed from a single component, e.g., an electrophile, which reacts with natural components of the tissue environment to produce a crosslinked polymeric network or scaffold within wound “W”.

Delivery device 130 is illustrated as a syringe including an outer shaft 132 including an inner channel, or chamber, 134 housing the second component 120 of the wound treatment system. An inner shaft, or plunger, 136 is slidably engaged within the inner channel 134 of the outer shaft 132 for driving the hydrogel material of the second component 120 disposed therein into wound “W”. The second component 120 is ejected from a tip 138 of the delivery device 130 by advancing the plunger 136 in the direction of the wound “W”. As described above, the second component 120 may be in a viscous form. The second component 120 fills the defect volume defined between the wound “W” and the first component 110. After placement of the second component 120 within wound “W”, the delivery device 130 is removed and the second component 120 is allowed to cure.

One may use a syringe for delivery of a single precursor, i.e., an electrophilic crosslinker, as described above, or a dual syringe or similar device to apply more than one precursor solutions, such as those described in U.S. Pat. Nos. 4,874,368; 4,631,055; 4,735,616; 4,359,049; 4,978,336; 5,116,315; 4,902,281; 4,932,942; 6,179,862; 6,673,093; and 6,152,943.

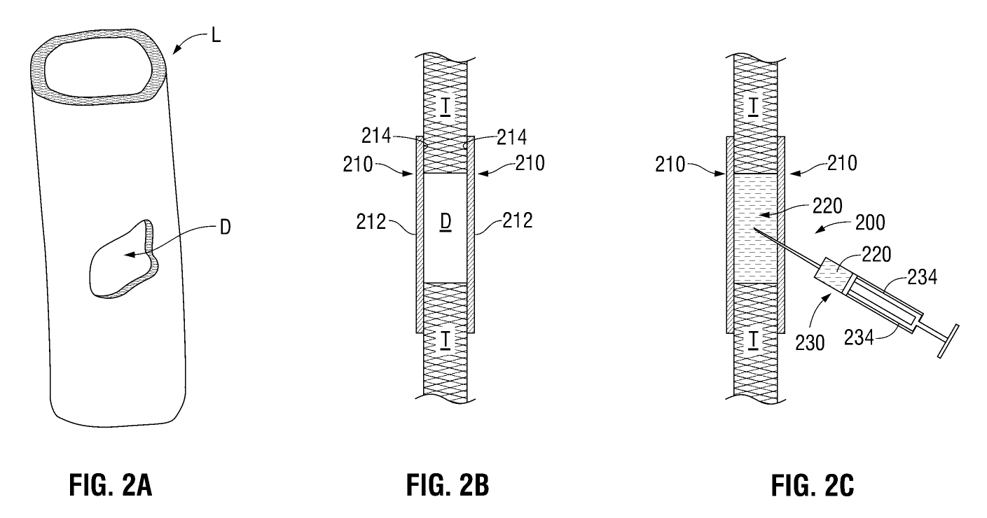

FIGS. 2A-2C illustrate a lumen “L” including a lumen defect “D”, e.g., an esophageal or bowel defect, being repaired by a wound treatment system 200 of the present disclosure. As illustrated in FIG. 2A, lumen defect “D” extends through the thickness of the tissue and requires placement of a first component 210 on each side of the defect “D” to define the defect volume as illustrated in FIG. 2B. The first component 210 is a polymeric sheet 212 including at least one functional group 214 on a surface thereof for adhering to tissue surrounding lumen defect “D”. A delivery device 230 loaded with second component 220 may be placed through a first component 210 as illustrated in FIG. 2C. The second component 220 is a hydrogel formed from two precursors that react to form a hydrogel. The delivery device 230 is similar to the delivery device 130 of the embodiment described above, except that the delivery device includes two inner chambers 234 for separately maintaining the two precursors of the second component 220 prior to delivery into lumen defect “D”. As illustrated, the two precursors mix immediately prior to delivery within the defect “D”. In other embodiments (not shown), the precursors may be ejected from separate tips to mix in situ. The second component 220 fills the defect “D” defined between the polymeric sheets 212. After placement of the second component 220, the delivery device 230 is removed and the second component 220 is allowed to cure. Alternatively, one polymeric sheet 212 of the first component 210 may be placed on a first side of a Lumen defect “D”. The delivery device 230 may be used to deposit the second component 220 in the defect and then a second sheet of the first component 210 may be placed on a second side of the defect to seal the second component between the polymeric sheets 212 of the first component 210.

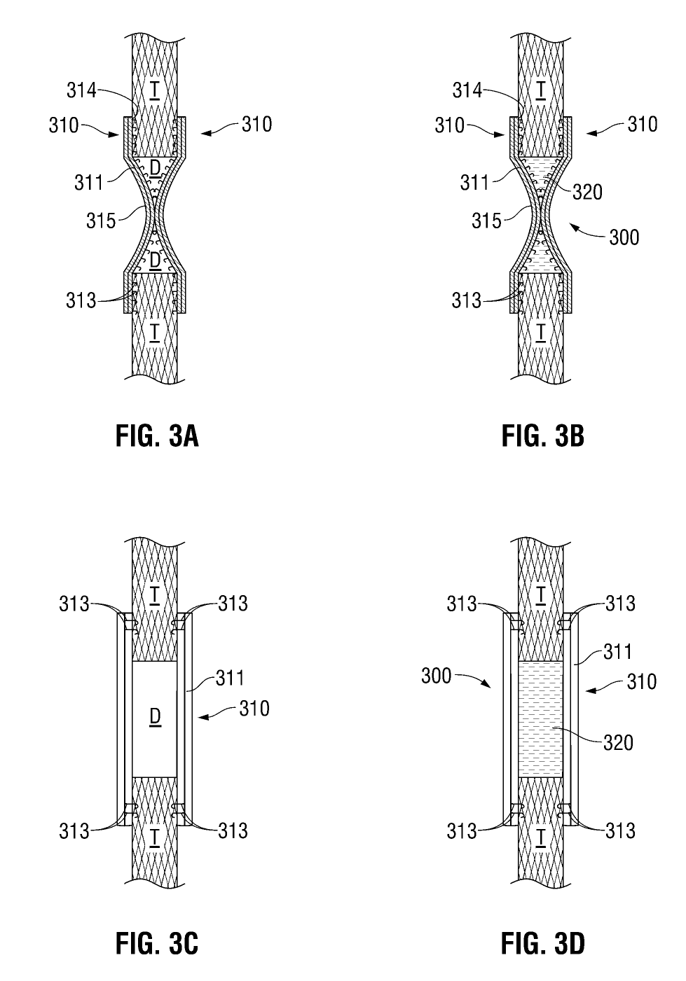

As illustrated in FIGS. 3A-3D, an alternate wound treatment system 300 includes a first component 310 in the form of a composite sheet including a mesh 311 having grip members 313 projecting from a surface thereof for mechanically engaging tissue “T”, embedded within a non-porous film 315. First component 310 may also include at least one functional group 314 disposed on a tissue facing surface. In the case of lumen defects “D”, as shown, opposing polymeric sheets 311 may bind to each other. A second component 320 may then be injected through one of the first components 310 to fill the defect “D” as illustrated in FIG. 3B.

As depicted in FIGS. 3A-3B, grip members 313 may, in embodiments, affix to tissue T as well as the opposite first component 310.

FIGS. 3C-3D show an alternate embodiment, where grip members 313 mechanically engage a portion of tissue “T” adjacent a lumen defect, helping to affix first component 310 of a composite mesh 311 on opposite sides of the lumen defect “D,” thereby permitting the introduction of second component 320 through first component 310 to fill lumen defect “D.” In this embodiment, grip members 313 only attach to tissue “T”.

The wound treatment system of the present disclosure may be provided as a kit. The kit may include a pre-formed, functionalized polymeric sheet that may be cut to size prior to use, a syringe, and hydrogel precursors. The hydrogel precursors may be pre-loaded into the syringe. In embodiments, a bioactive agent may be pre-mixed with the hydrogel precursors. For example, an amine reactive dry PEG-star pre-mixed with a growth factor, and a dry collagen may be provided in the kit. The hydrogel precursors may be solubilized at the time of surgery and loaded into separate chambers of a dual barrel syringe. In some embodiments, additional items may be provided in the kit, such as tools and vials to enable a clinician to collect cells or platelet rich plasma from the patient which may be mixed with the hydrogel precursors.

It will be understood that various modifications may be made to the embodiments disclosed herein. Therefore, the above description should not be construed as limiting, but merely as an exemplification of illustrative embodiments. Those skilled in the art will envision other modifications within the scope and spirit of the present disclosure. Such modifications and variations are intended to come within the scope of the following claims.

What is claimed is:

1. A method of treating tissue defects comprising:
placing a first polymeric sheet over one side of a lumen defect;
placing a second polymeric sheet over an opposite side of the lumen defect to define a defect volume; and
filling the defect volume with at least one hydrogel precursor including at least one reactive functional group.

2. The method of claim 1, wherein at least one of the first polymeric sheet and the second polymeric sheet is non-porous.

3. The method of claim 1, wherein at least one of the first polymeric sheet and the second polymeric sheet is porous.

4. The method of claim 1, wherein at least one of the first polymeric sheet and the second polymeric sheet is a composite of non-porous and porous layers.

5. The method of claim 1, wherein at least one of the first polymeric sheet and the second polymeric sheet includes a tissue facing surface including at least one pendant functional group for chemically binding the polymeric sheet to tissue.

6. The method of claim 5, wherein the at least one pendant functional group is selected form the group consisting of isothiocyanates, isocyanates, acyl azides, N-hydroxysuccinimide (NHS), sulfo-NHS esters, sulfonyl chlorides, aldehydes, glyoxals, epoxides, oxiranes, carbonates, arylating agents, imidoesters, carbodiimides, anhydrides, diazoalkanes, diazoacetyl compounds, carbonyldiimidazoles, disuccinimidyl carbonate, and combinations thereof.

7. The method of claim 1, wherein at least one of the first polymeric sheet and the second polymeric sheet includes a tissue facing surface including grip members for mechanically binding the polymeric sheet to tissue.

8. The method of claim 1, wherein the at least one reactive functional group of the at least one hydrogel precursor is an electrophilic group.

9. The method of claim 8, wherein the electrophilic group is selected from the group consisting of N-hydroxysuccinimides, sulfosuccinimides, carbonyldiimidazole, sulfonyl chloride, aryl halides, sulfosuccinimidyl esters, N-hydroxysuccinimidyl esters, succinimidyl esters, isocyanates, thiocyanates, carbodiimides, benzotriazole carbonates, epoxides, aldehydes, maleimides, imidoesters, and combinations thereof.

10. The method of claim 1, wherein the at least one reactive functional group of the least one hydrogel precursor is a nucleophilic group.

11. The method of claim 10, wherein the nucleophilic group is selected from the group consisting of —NH₂, —SH, —OH, —PH₂, —CO—NH—NH₂ and combinations thereof.

12. The method of claim 1, further comprising loading the at least one hydrogel precursor into a delivery device.

13. The method of claim 12, further comprising ejecting the at least one hydrogel precursor from the delivery device through at least one of the first polymeric sheet and the second polymeric sheet.

14. The method of claim 12, further comprising mixing a bioactive agent with the at least one hydrogel precursor.

15. The method of claim 12, further comprising:
loading a first hydrogel precursor into a first chamber of a delivery device; and
loading a second hydrogel precursor into a second chamber of the delivery device.

16. The method of claim 15, wherein the first hydrogel precursor is an electrophile and the second hydrogel precursor is a nucleophile.

17. The method of claim 1, wherein the first polymeric sheet, the second polymeric sheet, or both, further comprise at least one bioactive agent.
